(19) United States
(12) Patent Application Publication
(10) Pub. No.: US 2009/0287256 A1
(43) Pub. Date: Nov. 19, 2009

(54) VERTEBRAL FIXATION PLATE AND SCREW ASSEMBLY

(76) Inventor: Walter X. Loyola, Plano, TX (US)

Correspondence Address: STORM LLP, BANK OF AMERICA PLAZA, 901 MAIN STREET, SUITE 7100, DALLAS, TX, 75202, US

(21) Appl. No.: 12/121,192

(22) Filed: May 15, 2008

(51) Int. Cl. A61B 17/70; A61B 17/04

(52) U.S. Cl. 606/286; 606/246; 606/280; 606/301

(57) ABSTRACT

A plate and screw assembly for fixation of at least a first vertebrate to a second vertebrate in a spinal column is disclosed. The plate and screw assembly includes a plate member that extends over a portion of at least the first vertebrate. At least a first screw hole extends through the plate and is sized to receive a first screw. The first screw extends through the first screw hole to engage the cylindrical side section of the first vertebrate to secure the first vertebrate to the plate member. At least a second screw hole extends through the plate member and is sized to receive a second screw. The second screw extends through the second screw hole to engage one of the flat base sections of the second vertebrate.

### FIELD OF THE INVENTION

The invention relates to spinal fixation plates. More specifically, the invention relates to a plate and screw assembly for the fixation of vertebrae.

### BACKGROUND OF THE INVENTION

The human vertebral column, also called the spinal column, consists of thirty three irregular shaped bones known as the vertebral column. FIG. 1 depicts a vertebral column 100. The vertebral column has an anterior side 104 and a posterior side 108.

A top view of a vertebra 200 is depicted in FIG. 2. The vertebra 200 has a main body region 204 and a spinous process region 208. An opening 212 between the main body region 204 and the spinous process region 208 allows a spinal cord to pass.

FIG. 3A shows the main body regions 304 and 308 separated by an intervertebral disk 312. The intervertebral disk 312 resides in the intervertebral space 314. The main body region 304 (or 308) is drum-shaped having a cylindrical side section 306 and flat base sections 316 and 320.

FIG. 3B shows a perspective view of the main body region 304 with the cylindrical side section 306 and flat base sections 316 and 320. The intervertebral disk 312 is attached to the flat base sections of the main body regions. The intervertebral disk 312 cushions and softens the forces created by walking or jumping that might otherwise fracture the vertebrae.

Referring back to FIG. 2, the vertebra 200 moves relative to its adjacent vertebra, providing a range of flexibility to the vertebral column. The movement between the adjacent vertebrae is limited to prevent pressure on the spinal cord or bending of the spinal cord. Excessive pressure or bending of the vertebral column 100 may result in a disorder such as a ruptured or herniated disk or a bone spur. A ruptured or herniated disk may cause tissue to protrude from the disk to compress the spinal cord causing pain. Also, bone spur or osteophyte may impinge the spinal cord causing pain. Surgery is often required to correct problems with osteophytes and herniations of the intervertebral disks. Typically, during surgery one or more vertebrae are exposed and the intervertebral disk is removed, thus removing the protruding tissue or providing access for the removal of the bone osteophytes. The removal of the intervertebral disk causes the adjacent vertebrae to be disconnected. A spinal implant is inserted into the intervertebral space and a spinal fusion or fixation is performed to connect the adjacent vertebrae. The spinal fixation may be performed by a vertebral plate that connects two or more vertebrae.

FIG. 4 illustrates a section of a vertebral column 400 including vertebrae 404, 408, 412, 416, 420, and 424. Consider for example, that an intervertebral disk (not shown in FIG. 4) between the vertebrae 416 and 420 becomes herniated. Consequently, the herniated intervertebral disk is removed by surgery and an implant is inserted into the intervertebral space 432. Since the intervertebral disk connects the vertebra 420 to the vertebra 416, the removal of the intervertebral disk causes the vertebrae 420 to be disconnected or detached from the vertebra 416. Consequently, a vertebral fixation becomes necessary to re-attach the vertebra 420 to the vertebra 416 and to the vertebrae 404, 408 and 412.

A vertebral plate 440 is used attach the vertebra 420 to the vertebra 416 (and also to the vertebrae 412, 408). As shown in FIG. 4, the vertebral plate 440 extends at least partially over the cylindrical side sections 456 of the vertebrae 408-420. The vertebral plate 440 includes 4 pairs of screw holes, each screw hole being aligned to enable a screw to engage the cylindrical side section 456 of a single vertebra. The screws extend generally perpendicularly through the vertebral plate 440 to engage the cylindrical side section 456 of the vertebra.

FIG. 5 is a cross-sectional view of the vertebral column 400 including the vertebrae 404, 408, 412, 416, 420, and 424. As discussed before, the intervertebral disk (not shown in FIG. 5) between the vertebrae 416 and 420 is removed and an implant 430 is inserted into the intervertebral space 432. Consequently, the vertebra 420 became disconnected from the vertebra 416, requiring fixation. The vertebral plate 440 is used to connect the vertebra 420 to the vertebrae 416 (and also to the vertebrae 412, 408).

As shown in FIG. 5, the vertebral plate 440 is used to fixate the vertebra 420 to the vertebrae 416 (and also to the vertebrae 412, 408). The vertebral plate 440 receives a plurality of screws 444x each extending through the vertebral plate 440 to engage a vertebra. The screws are generally perpendicular to the vertebral plate 440 to enable the screws to extend through the cylindrical side of the vertebrae.

If subsequent to the fixation of the vertebra 420 to the vertebrae 416, an intervertebral disk 450 between the vertebra 420 and 424 is herniated or ruptured, removal of the intervertebral disk 450 may be necessary. If the intervertebral disk 450 is removed and an implant is inserted into the intervertebral space 452, the vertebra 424 will become disconnected from the vertebrae 420. Thus, a fixation will be necessary to connect the vertebra 424 to the vertebra 420. However, since the vertebral plate 440, which was implanted during a previous surgery, extends over at least a part of the cylindrical side section 456 of the vertebra 420, there is insufficient space remaining along the cylindrical side section 456 of the vertebra 420 to accommodate a new vertebral plate necessary to connect the vertebra 424 to the vertebra 420. As can be seen in FIG. 5, the edge of the vertebral plate 440 extends over at least part of the cylindrical side section 456 of the vertebra 420, leaving insufficient space remaining for another vertebral plate to extend over the cylindrical side section 456 of the vertebra 420 in order to fixate the vertebra 424 to the vertebra 420. Furthermore, even if space is available to accommodate a new vertebral plate to extend over the cylindrical side section 456 of the vertebra 420, an additional pair of screws into the cylindrical side sections 456 of the vertebra 420 may shatter or otherwise damage the vertebra 420. As will be appreciated, the cylindrical side section 456 of the vertebra 420 may be unable to sustain the impact from an additional pair of screws.

### SUMMARY

A plate and screw assembly for fixation of at least a first vertebrate to a second vertebrate in a spinal column is disclosed. The first and second vertebrae are adjacent to one another. Each vertebrae has a cylindrical side section and an upper and a lower flat base section. The plate and screw assembly includes a plate member that extends over a portion of at least the first vertebrate. At least a first screw hole extends through the plate and is sized to receive a first screw. The first screw extends through the first screw hole to engage the cylindrical side section of the first vertebrate to secure the first vertebrate to the plate member. At least a second screw hole extends through the plate member and is sized to receive a second screw. The second screw extends through the second screw hole to engage one of the flat base sections of the second vertebrate.

### BRIEF DESCRIPTION OF THE DRAWINGS

For a more complete understanding of the features and advantages of the present invention, reference is now made to the detailed description of the invention along with the accompanying figures and in which:

FIG. 1 depicts a vertebral column.

FIG. 2 depicts a top view of a vertebra.

FIG. 3A shows main body regions of a vertebra separated by an intervertebral disk.

FIG. 3B shows a perspective view of a main body region with the flat base sections.

FIG. 4 illustrates a section of a vertebral column.

FIG. 5 is a cross-sectional view of a vertebral column.

FIG. 6 illustrates a plate and screw assembly in accordance with one embodiment of the invention.

FIG. 7 illustrates a cross-sectional view of a plate and screw assembly in accordance with another embodiment of the invention.

FIG. 8 shows another view of the plate and screw assembly in accordance with one embodiment.

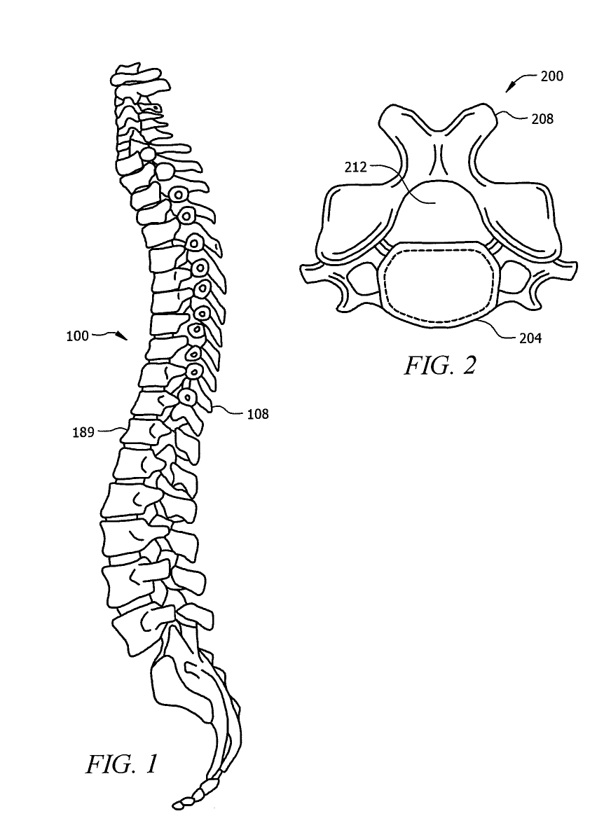

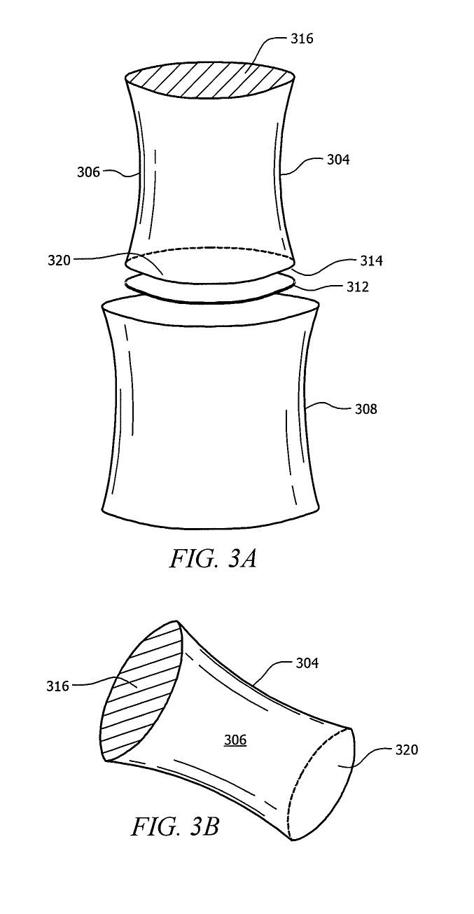

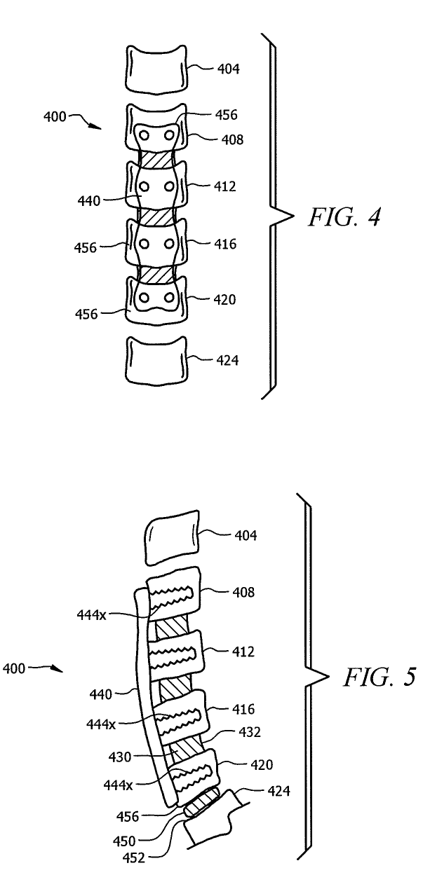

### DETAILED DESCRIPTION OF THE EMBODIMENTS

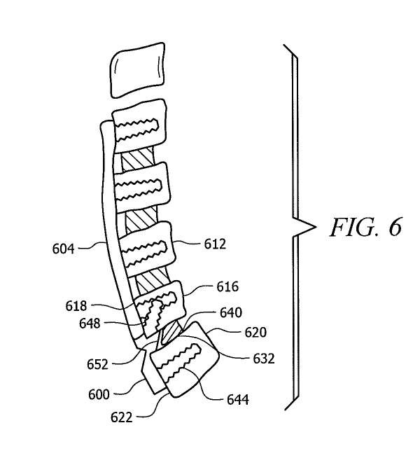

FIG. 6 illustrates a plate and screw assembly 600 in accordance with one embodiment. The plate and screw assembly 600 enables fixation of a first vertebra to a second vertebra when the second vertebra has inadequate space on its cylindrical side section to accommodate a conventional plate. As shown in FIG. 6, a conventional plate and screw assembly 604 is used to fixate a vertebra 616 to a vertebra 612. Since the conventional plate and screw assembly 604 extends over at least part of the cylindrical side section 618 of the vertebra 616, there is insufficient space remaining on the cylindrical side section 618 to accommodate a second conventional plate and screw assembly. However, the intervertebral disk (not shown in FIG. 6) in the intervertebral space 640 became ruptured or herniated, thus requiring removal of the intervertebral disk. As will be appreciated, the intervertebral disk in the intervertebral space 640 connects the vertebra 620 to the vertebra 616. Thus, the removal of the intervertebral disk from the intervertebral space 640 causes the vertebra 620 to be detached or disconnected from the vertebra 616.

An implant is inserted into the intervertebral space 640, and the plate and screw assembly 600 is used to attach or fixate the vertebra 620 to the vertebra 616. The plate and screw assembly 600 has a unique construction enabling the plate and screw assembly 600 to attach the vertebra 620 to the vertebra 616 without significantly extending over the cylindrical side section 618 of the vertebra 616. The plate and screw assembly 600 includes a first pair of screw holes configured to receive a first pair of screws 644 and a second pair of screw holes configured to receive a second pair of screws 648. The first pair of screws 644 extends generally perpendicularly through the plate and screw assembly 600 and engages the cylindrical side section 622 of the vertebra 620. As will be appreciated, the cylindrical side section refers to the side section of the main body region of the vertebra as discussed before. The second pair of screws 648 extends through the plate and screw assembly 600 at an angle to engage the flat base section 652 of the vertebra 620. As shown in FIG. 6, the angle at which the second pair of the screws 648 extend through the plate and screw assembly is a non-perpendicular angle in relation to the plate and screw assembly. In one example embodiment, the angle is 40 degrees, but other suitable values can be used. Thus, by extending at a non-perpendicular angle through the plate and screw assembly 600, the second pair of screws 648 can engage the flat base section 652 of the vertebra 620, thereby avoiding the cylindrical side section 618 of the vertebra 616. The angular orientation of the second pair of screws 648 enables the screws 648 to avoid the cylindrical side section 618 of the vertebra 616, thereby preventing damage to the vertebra 616. Since the cylindrical side section 618 of the vertebra 616 is already inserted with a pair of screws from the conventional plate and screw assembly 604, insertion of another pair of screws into the cylindrical side section of the vertebra 616 may cause the vertebra 616 to be shattered or otherwise damage. Accordingly, the plate and screw assembly 600 enables the second pair of screws 648 to engage the flat base section of the vertebra 616, thus attaching the vertebra 620 to the vertebra 616 without causing damage to the vertebra 616.

Furthermore, it is believed that since the second pair of screws 648 engages the flat base section 652 of the vertebra 616, the fixation of the vertebra 620 to the vertebra 616 is comparatively stronger than conventional fixation means as the flat base section 652 is structurally stronger than the cylindrical side section.

It will be appreciated that the plate and screw assembly 600 enables the fixation of the vertebra 620 to the vertebra 616 that would otherwise be difficult with the conventional plate and screw assembly. The plate and screw assembly 600 attaches the vertebra 620 to the vertebra 616 without engaging the cylindrical side section 618 of the vertebra 616. Especially in those circumstances where a conventional plate and screw fixates a first vertebra to a second vertebra, if a third vertebra is required to be fixated to the second vertebra but there is inadequate space to accommodate another conventional plate and screw assembly, the plate and screw assembly 600 enables the third vertebra to be fixated to the second vertebra.

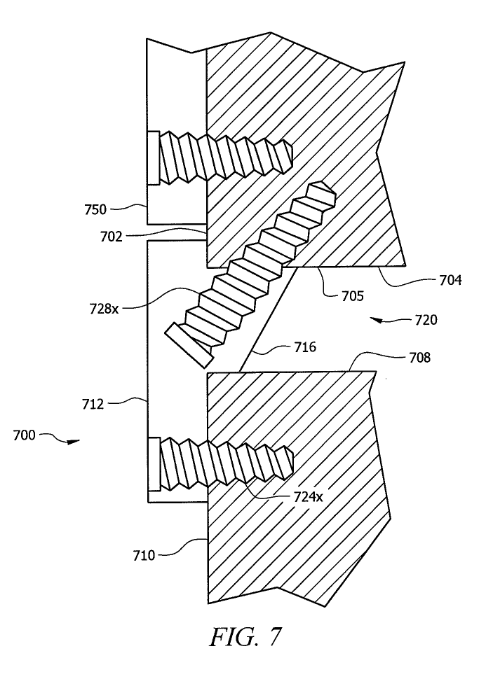

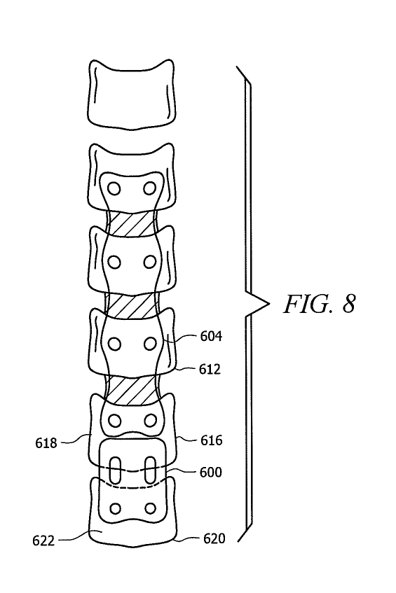

FIG. 7 illustrates a cross-sectional view of a plate and screw assembly 700 in accordance with another embodiment. The plate and screw assembly 700 enables the fixation of the vertebra 708 to the vertebra 704. As shown in FIG. 7, a conventional plate and screw assembly 750 has fixated the vertebra 704 to other vertebrae (not shown in FIG. 7). Since the conventional plate and screw assembly 750 extends over the cylindrical side section 702 of the vertebra 704, there is inadequate space remaining on the cylindrical side section 702 of the vertebra 704. Consequently, a second conventional plate and screw assembly is unsuitable to fixate the vertebra 708 to the vertebra 704.

However, the plate and screw assembly 700 due to its unique construction enables the vertebra 708 to be attached or fixated to the vertebra 704 in spite of the space limitation. The plate and screw assembly 700 includes a plate member 712 extending over at least a portion of the vertebrate 708. The plate and screw assembly 700 includes a cantilever member 716 attached to the plate member 712. The cantilever member 716 extends between the vertebrae 704 and 708 into the intervertebral space 720 to a selected length. By extending into the intervertebral space 720, the cantilever member 716 provides structural support to the vertebrae 704 and 708. A first pair of screws 724x extends perpendicularly through the plate member 712 and engages the cylindrical side section 710 of the vertebra 708. The first pair of screws 724x secures the vertebra 708 to the plate and screw assembly 700. The plate and screw assembly 700 includes a second pair of screws 728x extending through the plate member 712 and at least through a portion of the cantilever member 716. As will be appreciated, there are appropriate screw holes drilled into the plate member 712 and into the cantilever member 716 to receive the screws. The second pair of screws 728x engages the flat base section 705 of the second vertebrate 704.

The second pair of screws 728x is not perpendicular to the plate member 712, which enables the second pair of screws 728x to engage the flat base section of the vertebrate 704 without engaging the cylindrical side section 702 of the vertebrate 704. In one embodiment, the plate member 712 may extend over at least a portion of the cylindrical side section 702 of vertebrate 704 as shown in FIG. 7. It will be appreciated that in FIG. 7, a first pair of screw holes are drilled through the plate member 712 to enable the first pair of screws to extend perpendicularly through the plate member 712 to engage the cylindrical side section of the plate member 712. A second pair of screw holes are drilled through the plate member 712 and partially through the cantilever member 716 to enable the second pair of screws to extend through at an angle to engage the flat base section of the vertebra 708.

FIG. 8 shows another view of the plate and screw assembly 600. The plate and screw assembly 600 enables fixation of the vertebra 620 to the vertebra 616. A conventional plate and screw assembly 604 is used to fixate the vertebra 616 to a vertebra 612. Since the conventional plate and screw assembly 604 extends over at least part of the cylindrical side section 618 of the vertebra 616, there is insufficient space remaining on the cylindrical side section 618 to accommodate a second conventional plate and screw assembly. However, as discussed before, due to the unique construction of the plate and screw assembly 600, the vertebra 620 is attached to the vertebra 616.

While the compositions, structures, apparatus and methods of this invention have been described in terms of preferred embodiments, it will be apparent to those of skill in the art that variations may be applied to the compositions, structures, apparatus and/or methods and in the steps or in the sequence of steps of the method described herein without departing from the concept, spirit and scope of the invention. All such substitutes and modifications apparent to those skilled in the art are deemed to be within the spirit, scope and concept of the invention as defined by the appended claims.

What is claimed is:

1. A plate and screw assembly for fixation of at least a first vertebrate to a second vertebrate in a spinal column, the first and second vertebrae being adjacent to one another, each vertebrae having a cylindrical side portion and a base portion, comprising:
a plate extending over a portion of at least the first vertebrate;
at least a first screw hole extending through the plate and sized to receive a first screw, the first screw extending through the first screw hole to engage the cylindrical side portion of the first vertebrate to secure the first vertebrate to the plate; and
at least a second screw hole extending through the plate and sized to receive a second screw, the second screw extending through the second screw hole to engage the base portion of the second vertebrate.

2. The plate and screw assembly according to claim 1, wherein the first screw is positioned substantially perpendicular to the plate as it engages the first vertebrate.

3. The plate and screw assembly according to claim 1, wherein the second screw engages the base portion of the second vertebrate, and wherein the second screw is not perpendicular with respect to the plate.

4. The plate and screw assembly according to claim 1, wherein the plate has opposed first and second surfaces, the first surface facing the vertebrae.

5. The plate and screw assembly according to claim 1, wherein the second screw is positioned at an angle relative to the plate to enable the second screw to engage the base portion of the second vertebrate without engaging the cylindrical portion of the second vertebrate.

6. The plate and screw assembly according to claim 1, wherein the second vertebrate is secured to at least one other vertebrate in the spinal column.

7. The plate and screw assembly according to claim 1, wherein the plate extends over at least a portion of the second vertebrate.

8. An implant device for fixation of at least a first vertebrate to a second vertebrate in a spinal column, the first and second vertebrae being adjacent to one another, each vertebrae having a cylindrical side portion and a base portion, the implant device comprising:
a plate member extending over at least a portion of the first vertebrate;
a cantilever member attached to the plate member and extending between the first and second vertebrae to a selected length;
at least a first screw hole extending through the plate member and sized to receive a first screw, the first screw adapted to engage the cylindrical side portion of the first vertebrate to secure the first vertebrate to the plate member; and
at least a second screw hole extending through the plate member and at least through a portion of the cantilever member, the second screw hole being sized to receive a second screw and to enable the second screw to engage the flat base portion of the second vertebrate.

9. The implant device according to claim 8, wherein the first screw is positioned substantially perpendicular to the plate member as it engages the cylindrical portion of the first vertebrate.

10. The implant device according to claim 8, wherein the second screw is not perpendicular to the plate member to enable the second screw to engage the base portion of the second vertebrate without engaging the cylindrical portion of the second vertebrate.

11. The implant device according to claim 8, wherein the plate member extends over at least a portion of the second vertebrate.

12. A plate and screw assembly for fixation of at least a first vertebrate to a second vertebrate in a spinal column, the first and second vertebrae being adjacent to one another, each vertebrae having a cylindrical side portion and a base portion, comprising:
a plate extending over a portion of at least the first vertebrate;
first and second screw holes each extending through the plate and sized to receive a respective first and a second screw, the first and second screws each extending through the respective screw hole to engage the cylindrical side portion of the first vertebrate to secure the first vertebrate to the plate; and
third and fourth screw holes each extending through the plate and sized to receive a respective third and a fourth screw, the third and fourth screws each extending through the respective screw hole to engage the base portion of the second vertebrate.

13. The plate and screw assembly according to claim 12, wherein the first and second screws are positioned substantially perpendicular to the plate member as each engages the cylindrical portion of the first vertebrate.

14. The plate and screw assembly according to claim 12, wherein the third and fourth screws are not perpendicular to the plate member to enable the third and fourth screws to engage the base portion of the second vertebrate without engaging the cylindrical portion of the second vertebrate.

15. The plate and screw assembly according to claim 12, wherein the plate member extends over at least a portion of the second vertebrate.

16. An implant device for fixation of at least a first vertebrate to a second vertebrate in a spinal column, the first and second vertebrae being adjacent to one another, each vertebrae having a cylindrical side portion and a base portion, the implant device comprising:
a plate member extending over at least a portion of the first vertebrate;
a cantilever member attached to the plate member and extending between the first and second vertebrae to a selected length;
first and second screw holes each extending through the plate member and sized to receive a respective first and a second screw, the first and second screws each extending through the respective screw hole to engage the cylindrical side portion of the first vertebrate to secure the first vertebrate to the plate member; and
third and fourth screw holes each extending through the plate and at least through a portion of the cantilever member and sized to receive a respective third and a fourth screw, the third and fourth screws each extending through the respective screw hole to engage the base portion of the second vertebrate.

17. The implant device according to claim 16, wherein the first and second screws are positioned substantially perpendicular to the plate member as each engages the cylindrical portion of the first vertebrate.

18. The implant device according to claim 16, wherein the third and fourth screws are not perpendicular to the plate member to enable each of the third and fourth screws to engage the base portion of the second vertebrate without engaging the cylindrical portion of the second vertebrate.

19. The implant according to claim 16, wherein the plate member extends over at least a portion of the second vertebrate.
